(19) United States
(12) Patent Application Publication
(10) Pub. No.: US 2016/0000716 A1
(43) Pub. Date: Jan. 7, 2016

(54) METHOD OF TREATING VITAMIN B12 DEFICIENCY

(71) Applicant: Pharmaceutical Productions Inc., Miamia Springs, FL (US)

(72) Inventor: John McCarty, Miami Springs, FL (US)

(21) Appl. No.: 14/853,110

(22) Filed: Sep. 14, 2015

Related U.S. Application Data
(63) Continuation-in-part of application No. PCT/US2014/027412, filed on Mar. 14, 2014.
(60) Provisional application No. 61/782,246, filed on Mar. 14, 2013.

(51) Int. Cl. A61K 9/20; A61K 9/00; A61K 31/714

(52) U.S. Cl. CPC A61K 9/2013; A61K 31/714; A61K 9/2009; A61K 9/2018; A61K 9/2059; A61K 9/2054; A61K 9/0053; A61K 9/2095

(57) ABSTRACT

The present invention relates generally to Vitamin B12 pharmaceutical composition and method of using the same for the treatment of Vitamin B12 deficiency and the various disorders that are related to such deficiency. In particular embodiments, the present invention is directed towards treatment methods comprising sublingual or buccal administration of a Vitamin B12 composition useful in the practice of such treatment. The present invention features compositions that include one or more Vitamin B 12 compounds, propylene glycol, a solid adsorbent and a solid water-soluble excipient, wherein the Vitamin B 12 compounds are in a propylene glycol solution.

### CROSS-REFERENCE TO RELATED APPLICATIONS

This application is a Continuation-in-Part of International Application No. PCT/US2014/027412, filed on Mar. 14, 2014, which claims priority to U.S. Provisional Application Ser. No. 61/782,246, filed on Mar. 14, 2013. The contents of each are incorporated by reference herein in their entirety.

### FIELD OF INVENTION

The present invention relates generally to methods of treating Vitamin B 12 deficiency and a sublingual/buccal composition for such treatment.

### BACKGROUND OF THE INVENTION

Vitamin B-12 deficiency is very common. Large surveys in the United States and the United Kingdom disclosed that about 6% of those aged above or equal to 60 years are Vitamin B-12 deficient. Moreover, in developing countries like India this deficiency is much more common, starting in early life and persisting across the life span. A study of 441 middle-aged men in Pune (India) revealed that 67% of the men had low Vitamin B-12 concentration (<150 pmol/L). Of the urban middle class, 81% had low Vitamin B-12 concentration and vegetarians had 4.4 times higher risk of low Vitamin B-12 concentrations.

It is now well understood and accepted that Vitamin B-12 is an important and central factor in many body functions. It is necessary for normal metabolism of nerve tissue and is involved in protein, fat and carbohydrate metabolism. Vitamin B-12 is required for the synthesis and transfer of single carbon units such as the methyl group, and aids in the synthesis of methionine and choline, which are important lipotropic substances.

When the human body is healthy, the amount of Vitamin B-12 ordinarily absorbed into the blood by the intrinsic factor is about 2.5 to 3 micrograms per day. However, when the human body is not healthy and is suffering from pernicious anemia the body does not absorb adequate amounts of Vitamin B-12. The Vitamin B-12 deficiency manifests itself in human beings, most commonly, in motor and mental difficulties. The symptoms are rapid heartbeat, cardiac pain, and shortness of breath, edema of the face, general jaundice and intense brown discoloration around the small joints, weakness and fatigue. Neurological changes, such as peripheral neuritis, spinal cord changes, intermittent numbness and tingling in arms and legs, diminished tendon reflexes, unsteady gait, etc. may also occur.

Among its other functions, Vitamin B-12 is required for the formation of red blood cells and increases tissue deposition of Vitamin A by improving either carotene absorption or its conversion to Vitamin A. Vitamin B-12 is also closely related to the actions of four amino acids, pantothenic acid, and Vitamin C, and plays a part in reproduction and lactation. Additionally, Vitamin B-12 helps reduce the possibility of skin bruises and has been suggested as helpful in combatting alcoholism, diabetes mellitus, osteoarthritis, multiple sclerosis, certain mental diseases, and a number of other diseases and abnormalities.

Vitamin B-12, however, is a very complex Vitamin. It contains an atom of cobalt in its center and is a charged molecule with a high molecular weight. The structure is similar to that of hemoglobin with iron at its center and to chlorophyll with a central magnesium atom. It cannot be made synthetically, but must be grown, like penicillin, in bacteria or molds. Animal protein is virtually the only source in which Vitamin B-12 occurs naturally in substantial quantities. The human body cannot synthesize Vitamin B-12, and consequently, it must be obtained externally if there is a deficiency, that is, by diet.

In Vitamin B 12 deficiency, conversion of methylmalonyl-CoA to succinyl-CoA cannot take place, which results in accumulation of methylmalonyl CoA and aberrant fatty acid synthesis. In the other enzymatic reaction, methylcobalamin supports the methionine synthase reaction, which is essential for normal metabolism of folate. The folate-cobalamin interaction is pivotal for normal synthesis of purines and pyrimidines and the transfer of the methyl group to cobalamin is essential for the adequate supply of tetrahydrofolate, the substrate for metabolic steps that require folate. In a state of Vitamin B12 deficiency, the cell responds by redirecting folate metabolic pathways to supply increasing amounts of methyltetrahydrofolate. The resulting elevated concentrations of homocysteine and MMA are often found in patients with low serum Vitamin B12 and can usually be lowered with successful Vitamin B12 replacement therapy. However, elevated MM A and homocysteine concentrations may persist in patients with cobalamin concentrations between 200 to 350 pg/mL. Supplementation with Vitamin B12 during conditions of deficiency restores the intracellular level of cobalamin and maintains a sufficient level of the two active coenzymes: methylcobalamin and deoxyadenosylcobalamin.

The main causes of B-12 deficiency include lack of intrinsic factors and other intestinal factors (e.g. malabsorption), rare genetic disorders, conditions associated with gastric atrophy, infestation with tape worm, and inadequate intake. Therefore, it is necessary to overcome the deficiency of B-12 by supplementing with cyanocobalamin, hydroxocobalamin or methylcobalamin through various routes such as parenteral, nasal and oral.

Oral therapy is not suitable for patients lacking intrinsic factors, conditions associated with gastric atrophy, or infestation with tape worm. Further, to overcome such deficiency orally is extremely difficult even for those patients with intrinsic factor and good absorption since Vitamin B-12 does not become absorbed into the blood to any significant extent when taken orally, regardless of the amount. Berlin reported (H. Berlin et al, Acta Med. Scand. 184 247-258, 1968, and H. Hedstrand, Acta Med. Scand. 186 535-537, 1969) only approximately 1.2% of oral Vitamin B-12 is absorbed over rather a wide dosing range and such absorption rate is independent of the presence of intrinsic factor. Moreover, even insofar as the absorption of such a small quantity is concerned, there may be significant limitations such as a lack of hydrochloric acid, a lack of animal protein intake, or other gastro intestinal problems which create poor absorption capabilities.

WIPO patent application 2011/106378 A2 and 2009/1059188 A1 discloses the use of “SNAC” or Sodium-N-salicyloyl-8-aminocaprylate, Monosodium S—(N-salicyloylamino) octanoate, N-(salicyloyl)-8-aminooctanoic acid monosodium salt, monosodium N-{8-(2 phenoxybenzoyl)amino}octanoate, EDTA monosodium salt or sodium 8-[(2-hydroxybenzoyl)amino]octanoate in combination with Vitamin B12 to improve the oral bioavailability of Vitamin B12 in the treatment of Vitamin B12-deficient patients.

WIPO patent application 2008/099397 discloses the use of methylsulfonymethane as a transmucosal delivery enhancer which is claimed to enhance the delivery of a number of pharmaceutically active ingredients including Vitamin B 12. No specific embodiments however are disclosed for Vitamin B12.

WIPO patent application 2006/020291 A1 and 2007/030108 A2 discloses the use of mixtures of methylcobalamin, hydroxocobalamin, cyanocobalamin and adenosylcobalamin in various dosage forms and routes of administration including tablets, injectable, sprays and aerosols; however, no specific embodiments are disclosed for Vitamin B12.

Because of the extremely limited bioavailability of Vitamin B-12 when taken orally the preferred treatment process has to be in the form of Vitamin B-12 intramuscular (IM) injections. Such injections, however, have a number of significant drawbacks. First, injections are objectionable to administer because of the pain associated therewith. In this same regard, to many, the idea of injection treatments is inherently objectionable and offensive, and, consequently, there is a tendency not to proceed with the treatment. Additionally, as with any injection treatment process, needle abscess may occur and the treatment process is expensive.

In a newly-diagnosed Vitamin B12-deficient patient, normally defined as when serum cobalamin (Vitamin B12) levels are less than 200 pg/mL, daily EVI injections of up to 1,000 μg (1 mg) per day are given to replenish the body's depleted cobalamin stores. In the presence of neurological symptoms, following daily treatment, injections up to weekly or biweekly are indicated for 6 months before initiating monthly EVI injections. Once clinical improvement is confirmed, maintenance IM injection must be given for life.

Other routes of administration for Vitamin B 12, including nasal and oral sprays and transdermal patches have been considered in order to overcome the drawbacks of IM injection and poor oral absorption. However, sprays are less desirable because of inherent compliance issues such as improper manipulation of the actuator, swallowing of the dosage before absorption of the drug, and the restrictions on usage when the patient has sinus congestion or a head cold. This again leads to erratic and poor bioavailability. Therefore sprays are not the optimal route for routine Vitamin B12 administration.

WIPO patent application 86/05987 and 86/05988 disclose aerosol and nasal spray formulations for delivery Vitamin B 12.

WIPO patent application 2007/022345 discloses a nasally administered composition for delivery of Vitamin B12.

WIPO patent application 2012/056299 discloses an intranasal formulation which enhances the nasal absorption of Vitamin B 12.

WIPO patent application 2008/116004 A2 discloses a transdermal device for administering Vitamin B 12.

It can be appreciated from the foregoing that various internal and external factors may result in an individual experiencing a Vitamin B 12 deficiency. Currently, cyanocobalamin is available by prescription in an injectable form and as a nasal gel for the treatment of pernicious anemia. Over the counter preparations containing cyanocobalamin often include multivitamin, Vitamin B-complex, and

Vitamin B 12 supplements, which provide no benefit in treating patients lacking intrinsic factors, conditions associated with gastric atrophy, and malabsorption. It is clear that the present administration methods, in particular those using intravenous and nasal routes, make compliance difficult for any patient and particularly difficult for disabled, elderly and juveniles. Accordingly, it is desirable in the medical field to provide a means for the simple and reliable administration of Vitamin B 12 at appropriate dosages, over extended periods of time. One such alternative means may be administration via the sublingual/buccal route as disclosed herein.

### SUMMARY OF THE INVENTION

The present invention relates generally to methods of treating Vitamin B 12 deficiency and pharmaceutical compositions for such treatment.

One aspect of the invention is directed to a method for treating Vitamin B12 deficiency in a subject, comprising the steps of (a) preparing a pharmaceutical composition for sublingual/buccal administration containing (1) Vitamin B 12 and (2) at least propylene glycol, a pharmaceutically acceptable solid adsorbent and a water-soluble solid excipient (b) administering the pharmaceutical composition to the subject to effectively treat said Vitamin B12 deficiency.

Another aspect of the invention is directed to a pharmaceutical composition for treating Vitamin B12 deficiency in a subject, comprising (1) Vitamin B12 and (2) at least propylene glycol, a pharmaceutically acceptable solid adsorbent and a water-soluble solid excipient; wherein the dosage form is administered sublingually or buccally.

The contents of the patents and publications cited herein and the contents of these documents cited in these patents and publications are hereby incorporated herein by reference to the extent permitted.

### BRIEF DESCRIPTION OF THE DRAWINGS

The foregoing and other objects of the present disclosure, the various features thereof, as well as the disclosure itself may be more fully understood from the following description, when read together with the accompanying drawings in which:

FIG. 1 is a flow chart showing steps comprising the manufacture of a sublingual tablet containing a dose of 1 mg Vitamin B 12.

FIG. 2 is a graph depicting Vitamin B12 permeation over time for formulations of the present invention as compared to a commercially available B12 product designed for sublingual administration.

### DESCRIPTION OF PREFERRED EMBODIMENTS

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the disclosure described and claimed herein. The instant disclosure will govern in the instance that there is any inconsistency between the patents, patent applications, and publications and this disclosure.

The present invention provides formulations for sublingual and buccal administration, comprising Vitamin B-12 or any member of a group of cobalt-containing compounds known as cobalamins which include, but are not limited to cyanocobalamin, hydroxocobalamin, methylcobalamin, and 5-deoxyadenosyl-cobalamin. The cobalamins are mixed with propylene glycol and the resultant B-12/propylene glycol solution is added to a pharmaceutically acceptable solid adsorbent and a water-soluble solid excipient. Other excipients which aid in the performance or processing of the dosage form include pharmaceutically acceptable co-solvents or mixtures thereof, disintegrants, lubricants or combinations thereof. The invention also provides a process for preparing and method of administration of the disclosed formulation in the treatment of Vitamin B 12 deficiency.

In accordance with certain embodiments of the present invention, the composition comprises a pharmaceutically acceptable adsorbent selected from silica, microcrystalline cellulose, cellulose, silicified microcrystalline cellulose, clay, talc, starch, pregelatinized starch, calcium carbonate, calcium silicate, dicalcium phosphate, magnesium carbonate and mixtures thereof. In a preferred embodiment, the pharmaceutically acceptable adsorbent is silica, which is also called colloidal silicon dioxide.

Water-soluble solid excipients according to the invention are one or more of the following: sugars, polyols, saccharides, polysaccharides, dextrate, dextrins, dextrose, fructose, lactitol, lactose, erythritol, maltose, maltitol, maltodextrins, polydextrose, trehalose, mannitol, polyethylene glycols, isomalts, sorbitol, sucrose and xylitol. In one embodiment the water-soluble solid excipient is mannitol.

In an embodiment of the invention, Vitamin B 12 is mixed with propylene glycol. Suitable co-solvents include polyethylene glycol (PEG), e.g., PEG 400, PEG 200, PEG 300, PEG 600, or other molecular weight grades of PEG, ethanol, ethyl acetate, isopropyl alcohol, triacetin, triethyl citrate, tributyl citrate, substituted polyethylene glycols, bisabolol, glycerin, mineral oil, ethyl oleate, fatty acid esters, squalane, animal oils, vegetable oils, dimethyl isosorbide, hydrogenated vegetable oils, isopropyl myristate, isopropyl palmitate, glycofurol, terpenes, essential oils, alcohols, polyols, silicone fluids, and/or glycerides and combinations of such solvents. In one embodiment the co-solvent ethanol is used.

Other suitable excipients that might aid in the performance or to enhance processability, form, function, stability or aesthetic appeal of the formulation can be included in a composition according to the invention. Other excipients according to the invention are a buffering agent (such as phosphate, carbonate, tartrate, borate, citrate, acetate, and maleate buffers), colorant, flavoring, coating agent, binder, diluent, carrier, disintegrant, glidant, lubricant, opacifying agent, humectant, granulating agent, gelling agent, polishing agent, suspending agent, sweetening agent, anti-adherent, preservative, emulsifying agent, antioxidant, chelating agent, plasticizer, surfactant, tonicity agent, viscosity agent, enteric agent and coating, controlled-release agent and coating, wax, wetting agent, thickening agent, suppository base, stiffing agent, stabilizing agent, solubilizing agent, sequestering agent, mucoadhesive, ointment base, oleaginous vehicle, film-forming agent, essential oil, emollient, dissolution enhancer, dispersing agent, and/or cryoprotectant or combinations thereof.

In one embodiment, the pharmaceutical composition of the subject invention is provided as an oral dosage form for buccal or sublingual administration, e.g. films, lozenges, pills and tablets. In the following illustrative embodiments, the oral dosage form is provided as a tablet. In one embodiment, the pharmaceutical composition of the subject invention is provided as an oral dosage form for sublingual or buccal administration, e.g. films, lozenges, pills and tablets. In the following illustrative embodiments, the oral dosage form is provided as a tablet. In the following illustrative embodiments, the treatment is directed to subjects that had failed to respond to existing oral Vitamin B 12 treatment or are currently being administered Vitamin B 12 by EVI injection or nasal spray and wherein increasing the oral absorption and bioavailability, while shortening the onset of Vitamin B 12 action is provided.

It is understood by the skilled artisan, that use of the term “about” includes the range as stated, are within what is normally acceptable in the pharmaceutical industry. The US Pharmacopeia allows a plus and minus range of 10% in the assay for the active ingredient in most solid dosage forms. The Food and Drug Administration (FDA) has a published Guidances for changes in levels of common excipient classes that are considered unlikely to have any detectable impact on formulation quality and performance (Guidance for Industry: Immediate Release Solid Oral Dosage Forms Scale-Up and Post approval Changes: Chemistry, Manufacturing, and Controls, In Vitro Dissolution Testing, and In Vivo Bioequivalence Documentation). Under this Guidance the water-soluble solid excipient has an allowable change is +5%, for a disintegrant it is +1%, for a lubricant it is +1%. Although the Guidance is not specific for the complimentary lipophilic species, co-solvent or adsorbent and considering the range for the active is +10%, the value for these excipients should be no different than the active as their use in the formulation is directly dependent on the active's level.

As illustrated, tablets are used for the treatment and such tablets contain from about 0.05 mg to about 2 mg of Vitamin B 12, from about 1 mg to about 50 mg of a propylene glycol, from about 0.1 mg about 50 mg of a solid adsorbent, when included in a particular formulation, illustrated by, albeit not limited to silica, and from about 25 mg to about 500 mg of a water-soluble solid excipient, illustrated by, albeit not limited to, spray dried mannitol. In some instances the water-soluble solid excipient, illustrated by, albeit not limited to, spray dried mannitol, may function as the only solid adsorbent and as the water-soluble solid excipient in the particular formulation. In the case of certain formulations, an effective amount of a co-solvent may be necessary in order to enhance the transport of the active ingredient through the mucosal membrane. In such instances up to 25 mg per tablet is considered an effective amount to facilitate such transport, illustrated by, albeit not limited to ethanol.

In the illustrated embodiments, the tablet further contains at least one disintegrant and one lubricant. Although the disintegrant has been exemplified in the formulations in Table 1, 2 and 3 as sodium starch glycolate, it is nevertheless within the purview of this invention to substitute any functionally equivalent disintegrant, illustrated by, but not limited to, crospovidone, croscarmellose sodium, low-substituted hydroxypropyl cellulose, starch, microcrystalline cellulose and mixtures thereof. The content of the disintegrant is from about 0.5 mg to about 50 mg.

In the illustrated embodiments, the tablet further contains at least one lubricant. Although the lubricant has been exemplified in the formulations in Table 1, 2 and 3 as sodium stearyl fumarate, it is nevertheless within the purview of this invention to substitute any functionally equivalent lubricant, illustrated by, but not limited to, magnesium stearate, stearic acid, sodium lauryl sulfate, talc, polyethylene glycol, calcium stearate and mixtures thereof. The content of the lubricant is from about 0.1 mg to about 15 mg.

The present invention provides an unexpected increase in the rate and extent of drug absorption through the sublingual or buccal tissue. In a clinical setting this translates into increasing oral bioavailability and shortens the onset of drug action. One embodiment of the invention is prepared by dissolving Vitamin B12 into propylene glycol, with or without a co-solvent, and adsorbing this drug solution onto an acceptable pharmaceutical adsorbent, e.g. a silica and silicified microcrystalline celluloses. The liquid laden adsorbent is then combined with a water-soluble tablet diluent, a disintegrant and lubricant which is then compressed into a tablet for sublingual/buccal administration.

In the present invention Vitamin B 12 is in solution and this drug solution is combined with an adsorbent and then processed into a tablet for sublingual or buccal administration. In one embodiment of the invention it is the combination of the Vitamin B12 being in a propylene glycol solution and being adsorbed to a silica, which unexpectedly provides a significantly greater amount of drug transported across the sublingual mucosa and at a significantly greater rate. As a nutraceutical, Vitamin B12 is commercially available. In these commercially available products Vitamin B12 is in its solid state, as opposed to being in a solution as taught by the present invention, and is combined with other ingredients to make tablets for oral or sublingual administration. These prior art tablets suffer from a lack of sufficient permeation, which translates into a loss of bioavailability, delays the onset of action, and reduces the overall extent of action derived therefrom.

Accordingly, preparation of an exemplary composition according to the disclosure in the form of a tablet as disclosed by the instant invention, by dissolving Vitamin B12 into propylene glycol, with or without a co-solvent, and adsorbing this drug solution onto an acceptable pharmaceutical adsorbent, e.g. a silica and silicified microcrystalline cellulose, and adding the liquid laden adsorbent with a water-soluble tablet diluent, a disintegrant and lubricant and then processing into a tablet for sublingual administration. It is the combination of the Vitamin B12 being in solution and being adsorbed to silica which unexpectedly provides a significantly greater amount of drug being transported across the sublingual mucosa and at a significantly greater rate. This composition prepared in accordance with the method of the claimed invention thereby unexpectedly yields greater Vitamin B12 permeation, which translate clinically to greater bioavailability.

A method of manufacture for a tablet according to an embodiment of the subject invention for sublingual/buccal administration may employ any suitable method known in the art including, but not limited to, the addition of the Vitamin B 12 propylene glycol mixture with or without a co-solvent to premanufactured tablets, cold compressions with inert fillers and binders, direct tablet compression blends, direct powder blends, wet or dry granulations, molding, lyophilization, microencapsulation, freeze drying, spray-congealing, spray-drying, co-melt, spheronization, triturates, troching, powder layering, pelleting, encapsulation.

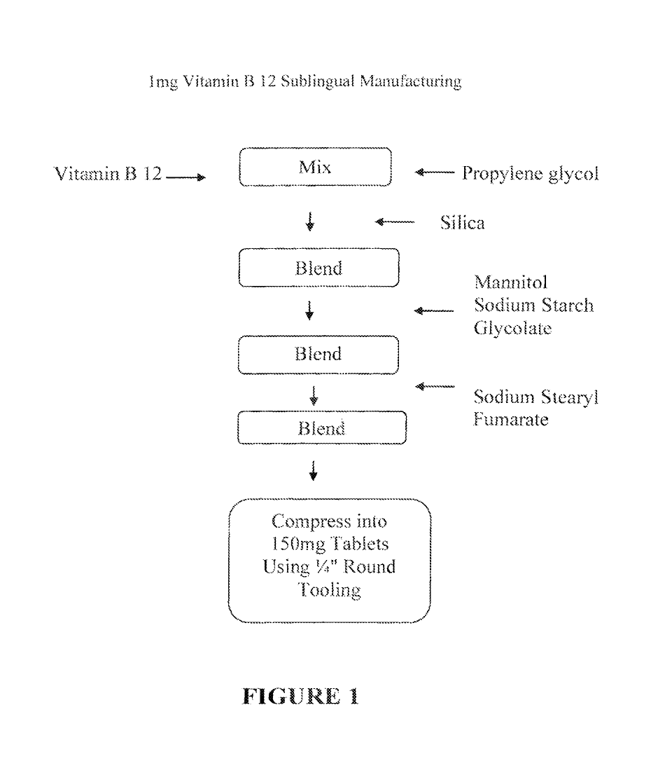

An exemplary method for the manufacture of a direct compression tablet of the formulation given in Example 1 is outlined below and is schematically diagramed in FIG. 1, and the steps outlined below:
STEP 1: Mix Vitamin B 12 and propylene glycol.
STEP 2: Blend the Vitamin B 12 and propylene glycol mixture from Step 1 with silica until homogeneous to form a silica adsorbent blend.
STEP 3: Add to the silica adsorbent blend from Step 2, mannitol and sodium starch glycolate and mix until homogeneous to form a further blend.
STEP 4: Add sodium stearyl fumarate to the further blend from Step 3 and blend until well lubricated to form a lubricated blend.
STEP 5: Compressing the lubricated blend from Step 4 into 150mg tablets using 1/4 inch round tooling.

The sublingual/buccal tablets may be packaged in such a manner as to aid in maintaining stability. Packaging methods and materials may include, but are not limited to, blister packaging in a foil/foil, foil/Acrylonitrile, foil/Polychlorotrifluoroethylene laminates for blister packaging or glass and plastic bottles.

In an embodiment, Vitamin B 12 buccal/sublingual tablet formulation according to the invention is useful in the treatment of pernicious anemia and other conditions brought on by a Vitamin B 12 deficiency. The typically treatment regimen starts by placing a Vitamin B 12 tablet under the tongue and leaving it undisturbed for about 5 to 15 minutes. The dosage range for this embodiment may vary from 0.05 to 2.0 mg depending on the therapeutic need.

The steps of dissolving the active ingredient, e.g. Vitamin B 12, to form an active ingredient-containing solution followed by contacting of the active ingredient-containing solution with the solid absorbent/adsorbent carrier whereby said active ingredient-containing solution is coated, absorbed or adsorbed onto said carrier are unique to the instant invention, and the carrying out of said steps are what allow for the formation of a unique solid dosage form which enables increased oral absorption and bioavailability while shortening onset of active ingredient action upon administration of the novel solid dosage form via the buccal or sublingual route.

Reference will now be made to specific examples illustrating the disclosure. It is to be understood that the examples are provided to illustrate preferred embodiments and that no limitation to the scope of the disclosure is intended thereby.

### EXAMPLES

The following experiments were performed using the Vitamin B12 composition of the invention.

### Example 1

### Drug Permeation

Drug permeation studies were performed using Epioral™ (see web site www.mattek.com), a fully differentiated, cultured oral mucosa as the relevant biological tissue. The graph below is the results obtained from sublingual permeation studies comparing GNC's 1 mg Vitamin B12 sublingual tablet to two formulations of a 1 mg Vitamin B12 sublingual tablet prepared according to the invention. Formulation FI is prepared per the invention using only propylene glycol to solubilize Vitamin B12 and formulation F2 uses propylene glycol along with the co-solvent ethanol. The compositions of formulations FI and F2 are given in Table 1 below.

TABLE 1
1 mg Vitamin B 12 Sublingual/Buccal Tablet Formulation

AMOUNT (mg tablet)

| INGREDIENT | FI | F2 |
|---|---|---|
| Vitamin B 12 | 1.00 | 1.00 |
| Propylene glycol | 14.00 | 4.77 |
| Ethanol | — | 0.30 |
| Silica | 9.60 | 4.00 |
| Mannitol | 132.00 | 92.10 |
| Sodium Starch Glycolate | 3.20 | — |
| LS Hydroxypropyl Cellulose | — | 20.11 |
| Sodium Stearyl Fumarate | 3.20 | 2.72 |
| Total Table Weight | 163.00 | 125.00 |

The 1 mg product marketed by GNC represents existing prior art. This product is a tablet designed to be placed under the tongue and allowed to dissolve before swallowing, i.e. sublingual administration. Further one of the main ingredients in the GNC tablet formulation is mannitol, which is the same tablet diluent used in the invention. Therefore comparisons are from similar formulations except for the inventive step of solubilizing Vitamin B12 in propylene glycol, with or without a co-solvent, and use of the adsorbent silica.

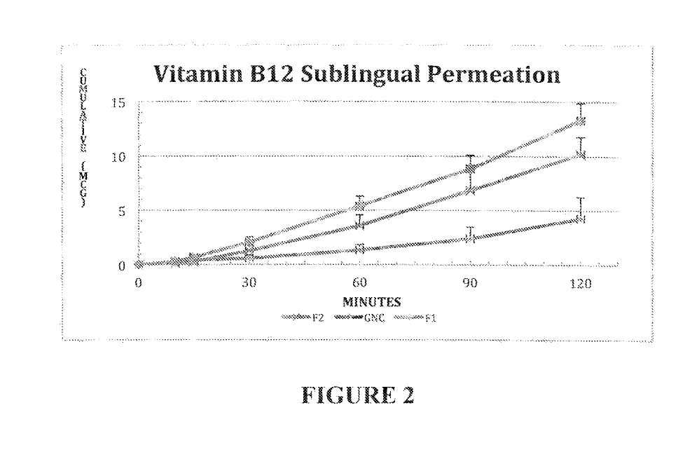

This study was conducted by mounting the Epioral™ tissue in a Franz celland the drug concentration was measured in the receiver solution over time. The tablets were placed on the donor side of the Franz cell and wetted with 1 ml of phosphate buffered saline at pH6.8, which was the same buffer used on the receiver side. Samples were taken from the receiver side of the Franz cell at the time points depicted in the graph of FIG. 2. Each formulation was run in triplicate, i.e., three Franz cells, and plotted as the mean value with a bar being used to show the sample standard deviation.

The permeation rates are calculated below:

Permeation rate between time points 30 and 120 minutes is calculated as:

INVENTION FI=10.21 mcg−1.27 mcg/90 minutes=0.001 mcg/minute INVENTION F2=13.32 mcg−2.05 mcg/90 minutes=0.125 mcg/minute

GNC=4.24 mcg−0.58 mcg/90 minutes=0.041 mcg/minute

Ratio of INVENTION FI to GNC's rates=0.1/0.41=2.44

Ratio of INVENTION F2 to GNC's rates=0.125/0.41=3.05

In conclusion, the data shows two and a half to three times the amount of Vitamin B12 permeated the sublingual tissue from the invention over the GNC's product and the rates was two and a half to three times greater. This translates clinically into significantly greater bioavailability of the invention over GNC's Vitamin B12 sublingual tablet and a more rapid onset which is important in sublingual delivery as residence time in the mouth is limited with this route of administration.

### Example 2

### Exemplary Tablets

In one embodiment, the invention provides a 1 mg strength Vitamin B12 sublingual/buccal tablet having a total tablet weight of about 150 mg, wherein the tablet comprises drug, a solid carrier, such as silica; a water soluble solid excipient, such as mannitol; a disintegrant, such as sodium starch glycolate; and a lubricant, such as sodium stearyl fumarate. In such an embodiment, Vitamin B 12 is mixed with propylene glycol. An exemplary formulation in accordance with the described formulation of this embodiment is provided in Table 2, below.

TABLE 2
1 mg Vitamin B 12 Sublingual/Buccal Tablet Formulation

| INGREDIENT | AMOUNT (mg tablet) |
|---|---|
| Vitamin B 12 | 1.00 |
| Propylene glycol | 11.00 |
| Silica | 9.00 |
| Mannitol | 121.50 |
| Sodium Starch Glycolate | 4.50 |
| Sodium Stearyl Fumarate | 3.00 |
| Total Tablet Weight | 150.00 |

In another embodiment, the invention provides 1 mg strength Vitamin B 12 sublingual/buccal tablet having a total tablet weight of about 150 mg. In this exemplary embodiment, Vitamin B 12 is mixed with propylene glycol and the co-solvent ethanol. An exemplary formulation manufactured for this embodiment in accordance with the subject invention is provided in Table 3, below.

TABLE 3
1 mg Vitamin B 12 Sublingual/Buccal Tablet Formulation

| INGREDIENT | AMOUNT Cms/tablet) |
|---|---|
| Vitamin B 12 | 1.00 |
| Propylene glycol | 11.00 |
| Ethanol | 2.00 |
| Silica | 10.00 |
| Mannitol | 118.50 |
| Sodium Starch Glycolate | 4.50 |
| Sodium Stearvl Fumarate | 3.00 |
| Total Tablet Weight | 150.00 |

In another embodiment, the invention provides a 0.1 mg strength Vitamin B12 sublingual tablet having a total tablet weight of about 160 mg. In this exemplary embodiment, Vitamin B12 is mixed with propylene glycol and added to spray dried mannitol, which functions as the water-soluble solid excipient and solid adsorbent. An exemplary formulation manufactured for this embodiment in accordance with the subject invention is provided in Table 4, below.

TABLE 4
0.1 mg Vitamin B 12 Sublingual Tablet Formulation

| INGREDIENT | AMOUNT (mg/tablet) |
|---|---|
| Vitamin B 12 | 0.1 |
| Propylene glycol | 1.5 |
| Mannitol | 150.9 |
| Sodium Starch Glycolate | 4.5 |
| Sodium Stearyl Fumarate | 3.0 |
| Total Tablet Weight | 160.0 |

The invention has been described with reference to various specific and preferred embodiments and techniques. However, it should be understood that many variations and modifications may be made while remaining within the spirit and scope of the invention.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A pharmaceutical composition containing a cobalamin in a solid dosage form for buccal or sublingual delivery, comprising:
a cobalamin;
propylene glycol into which the cobalamin is solvated; and
a solid adsorbent to which the solvated cobalamin is adsorbed,
the cobalamin being in solution in the solid dosage form of the pharmaceutical composition.

2. The pharmaceutical composition of claim 1, further comprising a co-solvent.

3. The pharmaceutical composition of claim 1, further comprising a water soluble excipient, a distintegrant, a lubricant, or a mixture thereof.

4. The pharmaceutical composition of claim 2, further comprising a water soluble excipient, a distintegrant, a lubricant, or a mixture thereof.

5. The pharmaceutical composition of claim 1, wherein the cobalamin comprises vitamin B12, cyanocobalamin, hydroxocobalamin, methylcobalamin, or 5-deoxyadenosyl-cobalamin.

6. The pharmaceutical composition of claim 2, wherein the cobalamin comprises vitamin B12, cyanocobalamin, hydroxocobalamin, methylcobalamin, or 5-deoxyadenosyl-cobalamin.

7. The pharmaceutical composition of claim 3, wherein the cobalamin comprises vitamin B12, cyanocobalamin, hydroxocobalamin, methylcobalamin, or 5-deoxyadenosyl-cobalamin.

8. The pharmaceutical composition of claim 1, wherein the solid adsorbent comprises microcrystalline cellulose, cellulose powder, silicified microcrystalline cellulose, silica, clay, talc, starch, pregelatinized starch, calcium carbonate, magnesium carbonate, or a mixture thereof.

9. The pharmaceutical composition of claim 2, wherein the co-solvent comprises ethanol, ethyl acetate, isopropyl alcohol, triacetin, triethyl citrate, tributyl citrate, substituted polyethylene glycols, bisabolol, glycerin, mineral oil, ethyl oleate, fatty acid esters, squalane, animal oils, vegetable oil, polyethylene glycols, hydrogenated vegetable oils, isopropyl myristate, isopropyl palmitate, glycofurol, terpenes, essential oils, alcohols, polyols, silicone fluid, glycerides, or a mixture thereof.

10. The pharmaceutical composition of claim 3, wherein the solid water-soluble excipient comprises a sugar, a polyol, a saccharide, a polysaccharide, a dextrate, a dextrin, dextrose, fructose, lactitol, lactose, erythritol, maltose, maltitol, a maltodextrin, a polydextrose, trehalose, mannitol, a polyethylene glycol, sorbitol, sucrose, xylitol, or a mixture thereof.

11. The pharmaceutical composition of claim 3, wherein the disintegrant comprises sodium starch glycolate, crospovidone, croscarmellose sodium, low-substituted hydroxypropyl cellulose, starch, microcrystalline cellulose, or a mixture thereof.

12. The pharmaceutical composition of claim 3, wherein the lubricant comprises sodium stearyl fumarate, magnesium stearate, stearic acid, sodium lauryl sulfate, talc, polyethylene glycol, calcium stearate, or a mixture thereof.

13. The pharmaceutical composition of claim 1, wherein:
the cobalamin is present in an amount of about 0.05 mg to about 2 mg;
the polyethylene glycol is present in an amount of about 1 mg to about 50 mg; and
the adsorbent is present in an amount up to about 50 mg.

14. The pharmaceutical composition of claim 2, wherein the co-solvent is present in an amount of about 25 mg.

15. The pharmaceutical composition of claim 3, wherein the water soluble excipient is present in an amount of about 25 mg to about 500 mg.

16. The pharmaceutical composition of claim 3, wherein the disintegrant is present in an amount of about 0.5 mg to about 50 mg.

17. The pharmaceutical composition of claim 3, wherein the lubricant is present in an amount of about 0.1 mg to about 15 mg.

18. A method for treating low Vitamin B12 levels, pernicious anemia, and other disease states for which Vitamin B 12 is an effective therapeutic, in a patient in need thereof, comprising: orally, buccally, or sublingually administering a therapeutically effective amount of the pharmaceutical composition of claim 1.

19. A method for treating low Vitamin B 12 levels, pernicious anemia, and other disease states for which Vitamin B 12 is an effective therapeutic, in a patient in need thereof, comprising: orally, buccally, or sublingually administering a therapeutically effective amount of the pharmaceutical composition of claim 2.

20. A method for treating low Vitamin B 12 levels, pernicious anemia, and other disease states for which Vitamin B 12 is an effective therapeutic, in a patient in need thereof, comprising: orally, buccally, or sublingually administering a therapeutically effective amount of the pharmaceutical composition of claim 3.

21. A method of manufacturing a pharmaceutical composition containing a cobalamin in solution in a tablet, comprising:
mixing the cobalamin with propylene glycol to form a cobalamin solution;
adsorbing the cobalamin solution to a solid adsorbent; and
compressing the cobalamin solution/adsorbent into a tablet,
wherein the cobalamin is in solution in the tablet.

22. The method of claim 21, further comprising the step of adding a disintegrant, a water-soluble excipient, a lubricant, or a mixture thereof to the cobalamin solution/adsorbent before the compression step.
